(19) United States
(12) Patent Application Publication
(10) Pub. No.: US 2007/0255184 A1
(43) Pub. Date: Nov. 1, 2007

(54) Disposable labor detection patch

(76) Inventor: Adnan Shennib, Dublin, CA (US)

Correspondence Address: GLENN PATENT GROUP, 3475 EDISON WAY, SUITE L, MENLO PARK, CA, 94025, US

(21) Appl. No.: 11/352,015

(22) Filed: Feb. 10, 2006

(51) Int. Cl. A61B 5/103

(52) U.S. Cl. 600/591

(57) ABSTRACT

The invention provides a low cost, fully integrated, disposable patch for the non-invasive monitoring of labor contractions. The patch monitors EMG bursts present on a pregnant woman's abdomen via a set of electrodes embedded in the invented patch. The contraction monitor patch is thin, flexible, and incorporates EMG amplifiers, a processor, a battery, and an indicator within. The indicator is activated when labor EMG patterns are detected. The labor detection patch is particularly suited for women with risk of premature delivery. The patch is unobtrusively and continuously worn, even during sleep and bathing. In another embodiment, the contraction monitor patch is used in hospitals during labor and delivery to monitor the status of contractions with a wireless link to an external monitoring unit.

### CROSS REFERENCE TO RELATED APPLICATIONS

This application is related to the U.S. patent application entitled Intrapartum Monitor Patch, filed jointly with this application, and co-pending patent application Ser. No. 10/866,378. These applications are incorporated herein in their entirety by this reference herein.

### BACKGROUND OF THE INVENTION

1. Technical Field

The invention relates to non-invasive monitoring of a pregnant female. More particularly, the invention relates to the detection of labor during pregnancy.

2. Description of the Prior Art

Labor contractions are the periodic tightening and relaxing of the uterine muscle, the largest muscle in a woman's body. During a contraction, the abdomen becomes hard to the touch. In the childbirth process, the work of labor is done through a series of contractions. These contractions cause the upper part of the uterus (fundus) to tighten and thicken while the cervix and lower portion of the uterus stretch and relax, helping the baby pass from inside the uterus and into the birth canal for delivery. Contractions occur early in the pregnancy but are generally weak, irregular and often unfelt until the seventh or eighth month of pregnancy. Little or no change occurs in the cervix during these pre-labor contractions, sometimes referred to as false labor, or Braxton-Hicks contractions. At full term, generally defined as beyond 37 weeks of gestation, more intense and regular contractions occur to assist the mother in the normal delivery of the baby. The duration and intensity of contractions, referred to herein as contraction patterns, vary widely according to the condition and stage of pregnancy. These patterns dramatically change during active labor with its four stages: the first ending with full dilation of the cervix, the second ending with the birth of the baby, the third ending with the delivery of the placenta, and the final stage following the delivery of the baby and placenta.

For variety of reasons including congenital disease, mother's lifestyle, multiple gestation, complications, and other unknown causes, many babies are born prematurely (preterm). In the U.S. alone, approximately 1300 babies, or approximately 10%, are prematurely born every year. Most are too weak and too sick to go home and may spend weeks or even months in a neonatal intensive care unit. The consequences of preterm birth are serious and include the demise of the newly born and increased risk of impaired development for the survivors. Despite numerous medical advances, the short-term direct cost of neonatal heath care of premature births is extremely high and represents a significant percentage of the total health care cost. The indirect financial cost to the family, employer, healthcare system, and the society in general is also very high. The social and emotional costs of preterm births are simply immeasurable.

When premature labor is detected, the goal is to stop the premature labor and prevent the baby from being delivered before its full term. A first recommendation for the woman experiencing premature contractions may be to lie down with feet elevated and to drink fluids. If contractions continue or increase, medical attention should be sought. In addition to bed rest, medical care may include intravenous fluids and oral or injectable drugs such as terbutaline sulfate, ritodrine, magnesium sulfate, or nifedipine. These and other tocolytic treatments are generally more effective when preterm labor is detected early. Some women may need continued medication to prevent pre-term contractions. In some cases, the detection of preterm contractions may reveal the need for preterm delivery to improve the odds of survival for the baby.

In general, if premature labor is managed successfully, a pregnancy may continue normally for the delivery of a healthy infant. Once a premature labor occurs during the pregnancy, the mother and fetus need to be monitored regularly because premature contractions are likely to occur again. Women with high-risk pregnancies are particularly at risk of premature labor leading to premature delivery. Delaying the delivery of a premature infant for even one week not only improves the odds of healthy survival for the baby but also reduces health care expenditures by tens of thousands of dollars according to industry reports.

Upon pregnancy, the expectant mother is often instructed to self monitor her own contractions by palpitating the uterus area and checking for its hardness during contraction episodes. When contraction patterns are consistent and regular, the mother is typically advised to contact her health care provider.

Predicting or detecting the occurrence of premature labor is sometimes difficult for the mother or the medical staff. Premature labor contractions are sometimes painless and without any symptoms. For others, contractions may be confused with other abdominal symptoms, such as intestinal cramps and backache.

Various instrumentations are used for the objective assessment of contraction during labor. Commercially available non-invasive options generally rely on toco transducers (tocodynamometer), which are held against the abdomen by a belt or a harness and connected to an external monitor for displaying pressure change patterns during contractions. The intensity and duration of a contraction is typically observed along with fetal heart rate (FHR) during the fetal-maternal monitoring process. However, this non-invasive method does not always adequately detect contractions and thus necessitates the use of an intrauterine pressure (IUP) catheter. This invasive alternative relies on a pressure sensing catheter introduced vaginally into the uterus after the cervix is dilated. The pressure sensor at the tip of the catheter responds to uterine contractions and relays pressure signals to the external monitor via the connecting cable. A major disadvantage of IUP method is that it can be used only after membrane rupture and it requires the presence of an obstetric specialist.

Current contraction sensing instruments are generally bulky and difficult to operate and that thus are limited to clinical settings with trained personnel to operate them. Even with recent advances in electronic miniaturization and microprocessor applications, the cost and inconvenience of current instruments limit their application to specialized clinical settings, such as gynecology offices and hospitals. For home applications, portable instruments can be used by the expectant mother but generally are limited to those expectant mothers with high-risk pregnancies.

U.S. Pat. No. 6,440,089 by Shine discloses a uterine contraction detector, shown as a desktop unit, with a method of determining the frequency of contractions, trending the frequency data, and generating a real-time graphical representation of the determined frequency.

U.S. Pat. No. 6,169,913 by Hojaiban et al. discloses an apparatus and method of sensing uterine activity by sensing changes in blood volume in the abdominal wall. A particular method disclosed involves detecting reflected light, indicating its absorption by hemoglobin present in abdominal blood vessels.

The occurrence and progress of labor can be assessed non-invasively using electromyogram (EMG) signals from the uterus. Uterine EMG bursts, also referred to sometimes as electrohysterography (EHG), characterize uterine contractile events during pregnancy. This activity is generally low but rises dramatically during labor. Prediction of term and preterm delivery with transabdominal EMG electrodes has been investigated by several researchers, showing a correlation between EHG and IUP for both preterm and term labor.

Nathanielsz in U.S. Pat. No. 4,967,761 discloses a method of characterizing myometrial activity to distinguish term and preterm labor from contractures.

U.S. Pat. No. 6,134,466 to Rosenberg discloses a method and system, shown as desktop apparatus, for detecting EMG signals by analyzing the average frequency of each contraction and indicating true labor when the last discriminant exceeds a threshold value.

These and other prior art instruments and methods are not only expensive and difficult to operate, but they are cumbersome for self monitoring, particularly during sleep when considering the physical aspect and profile of these systems.

An object of the invention is to detect labor with an unobtrusive device that is worn continuously and conveniently by an expectant mother.

A further objective of the invention is to provide a low cost labor sensor that is fully automatic and integrated.

A further objective is to provide a preterm labor sensor for females with risk of premature pregnancy.

A further objective is to develop a labor monitor, which minimizes supervision and intervention by medical personnel.

A further objective is to provide a real-time labor monitor with an integrated indicator.

A further objective is to provide a non-obtrusive contraction monitor for hospital use to indicate the occurrence and progression of labor non-invasively.

### SUMMARY OF THE INVENTION

The invention provides a low cost patch for the non-invasive detection of labor for a pregnant woman. The patch is adhered to the abdomen area of an expectant mother for continuous and automatic monitoring of uterine electromyogram (EMG) signals present on the abdomen area. Uterine myometrial activity patterns, particularly frequency and intensity patterns of EHG, are continuously monitored and analyzed by the invented patch in search of a labor detection criteria. Signal detection is enhanced by the proper placement of electrodes and advanced signal processing. In addition to detecting labor events, the patch in one embodiment is employed to predict delivery, particularly for women at risk of preterm delivery.

The contraction monitor patch is thin, flexible, and incorporates electrodes, biopotential amplifiers, a processor, a memory, a battery, and an indicator. The indicator is activated when a predetermined criteria of EMG patterns is detected, for example when contractions occur at a rate of four times per hour or more. In the preferred embodiment, the monitor patch is disposable, and is thus discarded after battery depletion, detecting labor event, or after delivery. Although particularly useful for monitoring women with risk of premature delivery, the simplicity and low cost aspect of the invented patch allow for use by all pregnant women. In another embodiment, the monitor patch is also used to indicate the progression of labor.

The labor sensor patch is suited for long-term wear, lasting several days for detecting premature contractions in risk pregnancies. In this application, the patch is worn continuously, even during sleep and showering, and is thus made durable and waterproof, while being flexible and unobtrusive, for inconspicuous wear underneath clothing. Alternatively, the EMG sensor patch can be used for short-term applications, such as during labor and delivery in maternity wards of hospitals.

The monitor patch can also incorporate fetal heart rate monitoring as disclosed in the pending application Ser. No. 10/866,378. The sensors in the preferred embodiment of the invented patch comprise biopotential electrodes with biopotential amplifiers. However, electromechanical elements including piezoelectric and miniature toco transducers may be incorporated into the invented patch to detect abdominal pressure changes during labor.

In another embodiment, the fetal monitor patch is wirelessly programmable using an external programmer for programming detection criteria according to the individual pregnant female. The patch can also record contraction data in memory for subsequent retrieval. Wireless transmission of real-time or recorded contraction data to a monitoring station is incorporated in a hospital embodiment to provide continuous monitoring of myometrial activity during labor and delivery.

In the hospital embodiment of the invention, EMG signals detected during labor can be electronically translated by the proper interface to produce an electrical signal that emulates pressure-representative signals produced by a standard toco transducer, or an IUP transducer. This emulation technique allows the invented patch to interface with standard fetal-maternal monitors widely available, thus producing a familiar display and monitoring process with existing equipment.

### BRIEF DESCRIPTION OF THE DRAWINGS

FIG. 1 is a frontal view of a labor monitor patch placed on the abdomen of an expectant mother, in which the patch is vertically elongated with EMG electrodes for sensing myometrial activity;

FIG. 2 is detailed view of the vertically myometrial monitor patch of FIG. 1 showing the major internal components;

FIG. 3 is a cross section view of the monitor patch in FIG. 2;

FIG. 4 is a detailed cross section view of a section of the sensor patch of FIG. 2, showing the various layers including a metal foil layer;

FIG. 5 shows a rectangular embodiment of the myometrial monitor patch having three electrodes;

FIG. 6 shows a five-electrode embodiment placed on the abdomen of an expectant mother with EMG sensing for labor activity monitoring and ECG sensing for fetal and maternal heart rate monitoring;

FIG. 7 is a schematic diagram of the electronic assembly within the myometrial activity sensor patch, showing audible and visual indicators;

FIG. 8a shows a composite ECG signal with fetal QRS and maternal QRS components;

FIG. 8b shows an extract QRS complex of the fetal ECG;

FIG. 9 shows a fetal monitor patch placed on the side of the abdomen;

FIG. 10 shows an embodiment of the fetal monitor patch having five biopotential electrodes;

FIG. 11 shows a labor monitor patch having a wireless interface to standard fetal monitor instrument with a wireless interface device in proximity to the patch device;

FIG. 12 shows a block diagram of the wireless interface device with wireless receiver and signal conditioner to provide simulated signal to standard fetal monitoring instrument; and

FIG. 13 shows the invented patch equipped with acoustic transducers for transferring myometrial data acoustically over the telephone.

### DETAILED DESCRIPTION OF THE INVENTION

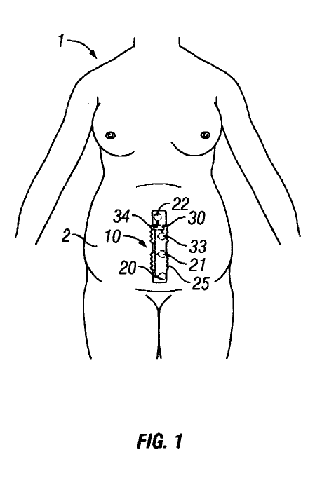

The invention, shown in various embodiments of FIGS. 1-13 is a non-invasive contraction monitor 10 in the form of a patch placed on the abdomen area 2 of an expectant mother 1. The electronic patch device 10 is thin and flexible for unobtrusive, continuous wear.

Referring to the embodiment of FIGS. 1-3, the patch device 10 comprises EMG electrodes, 20, 21, and 22. The device 10 also comprises an electronic assembly 30 including a biopotential amplifier 31, a processor 32, and a power source 33. The processor 32 is typically a digital signal processor for performing numerical computation from data obtained from an analog-to-digital converter 36. The power source 33 in the preferred embodiments is a primary battery having long shelf life.

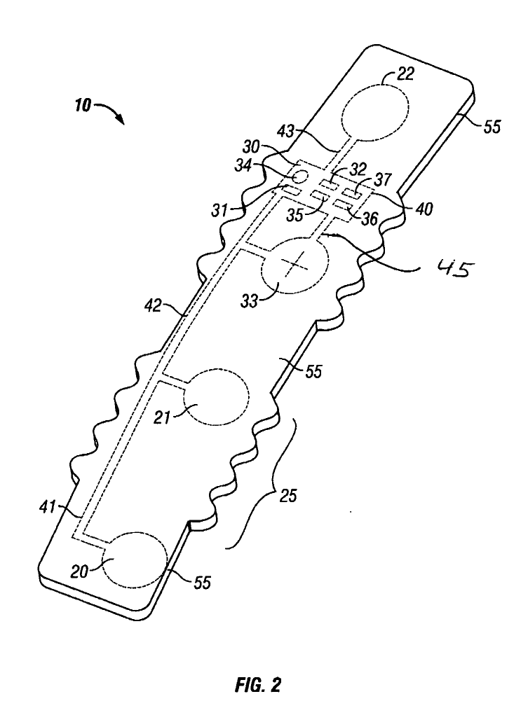

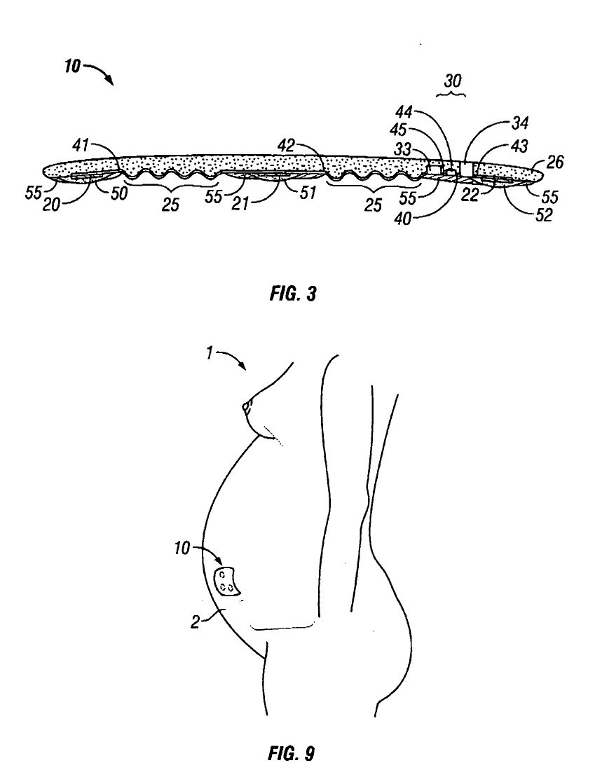

In a more detailed view of the device shown in FIGS. 2-5, the electronic assembly 30 is mounted on a flexible circuit substrate 40 with trace extensions 41, 42, 43, and 45 connecting the electronic assembly 30 to electrodes 20, 21, 22 and the power source 33, respectively. Conductive adhesive films 50, 51, and 52 cover metal electrodes 20, 21, and 22, respectively. Conductive adhesive films 50, 51, and 52 contact the skin directly to conduct surface EMG potentials to the amplifier 31. A non-conductive adhesive 55 provides an overall adhesive to secure the patch device 10 to the body. The device 10 also comprises a thin substrate 26 (FIG. 3-5) for providing structural support. The substrate 26 is made of soft flexible sheath material, such as polyurethane, cotton, or other material used in medical patch applications. The thickness of the patch device 10 is preferably in the range of 1.5 to 2.5 mm, but no more than 3.5 mm.

The patch assembly 10 may comprise as few as two electrodes or as many as five or more electrodes, depending on the desired application. Two to three electrodes are sufficient for basic monitoring applications, where only basic features of EMG signal are required. Additional electrodes and sensors (not shown) can be incorporated for determining additional vital signs, such as fetal and maternal heart rate, which can be extracted from a composite ECG biopotential signal (FIG. 8A) also present on the abdomen of a pregnant woman. The extraction of fetal ECG (FIG. 8B) from composite ECG is disclosed in detail in patent application Ser. No. 10/866,378, which is incorporated herein in this entirety by this reference thereto.

FIGS. 1-4 show an elongated patch arranged in a vertical electrode configuration. FIG. 5 shows an alternate three-electrode configuration, the patch is rectangular in shape, having a single upper electrode (E_{U}), and two lower electrodes, E_{R}, E_{L} for placement on the right and left sides of the lower abdomen.

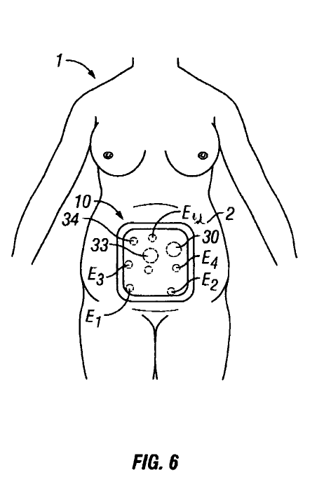

FIG. 6 shows a five-electrode embodiment, having an upper electrode E_{U} and four abdominal electrodes E₁, E₂, E₃ and E₄, for EHG and ECG monitoring. The biopotential electrodes of the invention detect both EHG and ECG signals using the same set of electrodes, thus providing an integrated electronic solution to labor and vital sign sensing. This is in stark contrast to electromechanical sensors found in reusable sensors used in clinical setting including toco and ultrasound transducers.

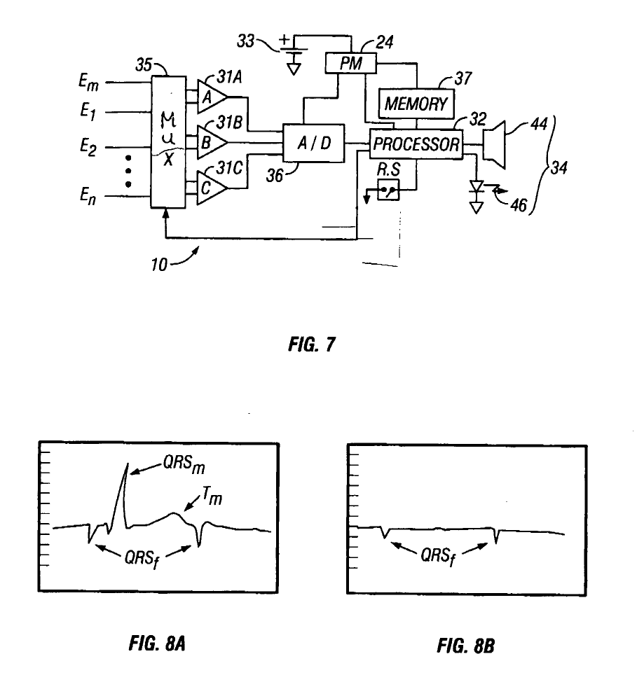

The multi-abdominal electrode configuration is also useful in applications to minimize the effects of artifacts present on the abdomen and for ensuring continuous EMG and ECG signal detection. Multiple electrodes minimize the effect of fetal position and movements. This is partially accomplished by the application of a multiplexer (MUX, 35; FIG. 7), whereby the appropriate electrodes can be paired as a differential input to biopotential amplifiers 31A, 31B, and 31C. Because the multiplexer 35 is under the control of the processor 32, selection of electrode pairs can be dynamically performed in real-time to obtain the desired biopotential signal. Alternatively, the application of adaptive signal processing for signal enhancement and cancellation of undesired signal can be accomplished digitally with a fixed set of biopotential amplifiers. Thus, an analog multiplexer is not required.

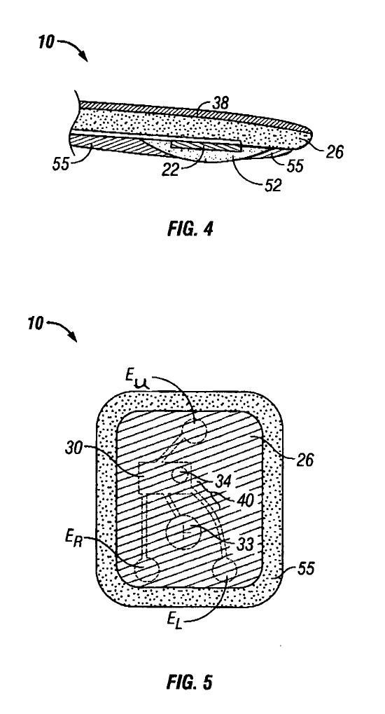

Various filtering methods are known in the field of signal processing and particularly pertaining to EMG and ECG signals. Filtering is not only necessary for removing undesired biopotential signals such as ECG and muscular EMG for obtaining EHG, but also for filtering out electromagnetic interference (EMI). To minimize interference further, a metal foil 38 (FIG. 4) is preferably provided, either entirely over the substrate 26 or selectively over electronic traces and components sensitive to interference.

In a preferred embodiment of the invention, the patch is used to detect early signs of premature contractions for mothers with risk of premature delivery. By continuously sensing of EMG patterns on the abdomen of a pregnant woman, true labor and adverse contraction conditions can be detected and differentiated from ordinary myometrial contractures including false labor. For example, the intelligent patch of the invention may be programmed to detect and indicate the occurrence of labor once contractions occur at a rate of four times per hour. When this occurs, the patch alerts the mother via the integrated indicator 34, which may be of any form perceptible by the expectant mother. The pregnant woman can then alert her medical provider for intervention, which may include the administration of a tocolytic agent to halt or delay a premature delivery. The indicator 34 may also be used to indicate the progression of labor from an early stage through later stages. An indicator in the form of an alarm transducer can be activated during a labor event detected by the monitor device 10. The indicator transducer 34 may be in the form of an audible transducer (44, FIG. 7), such as a buzzer or a speaker; or it may be in the form of visual display 46, such as a light emitting diode (LED) or a liquid crystal display (LCD). In the case of a visual indicator, each stage of labor can be indicated by text or multicolor LEDs showing, for example, green for early labor, orange for first stage active labor, and red for second stage active labor.

Another example of an indicator transducer is a vibrating element for imparting tactile sensations for the mother. The indicators may also be used to indicate other monitored parameters, such as fetal and maternal heart rate.

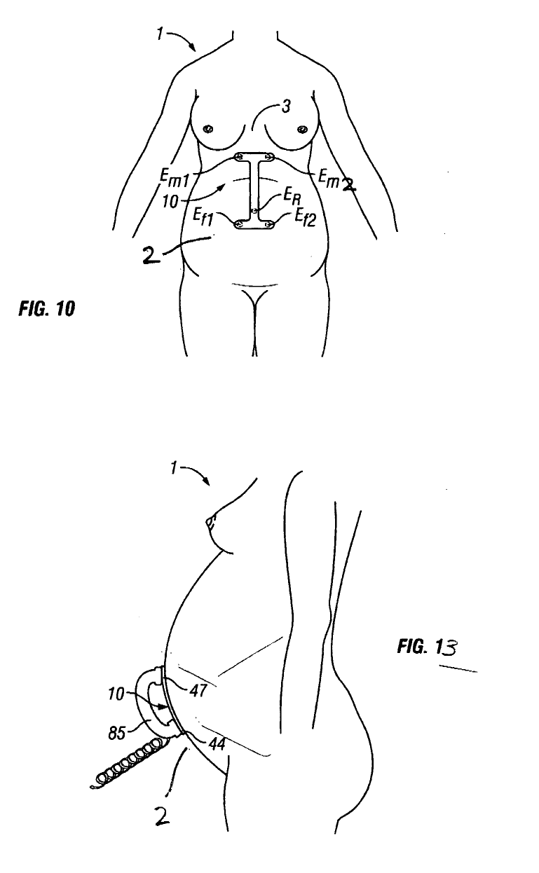

FIG. 9 shows an embodiment placing the labor monitor patch device 10 on the side of the abdomen. Other configurations of the invented patch include five electrodes configured in an “H” format, as shown in FIG. 10. In this configuration, electrodes Eₘ₁ and Eₘ₂ are also used for jointly receiving EMG and maternal ECG, along with abdominal electrode E_{f1} and E_{f2} for jointly receiving EMG and fetal ECG contaminated with maternal ECG component. A reference electrode E_{R} is used as a reference node for maternal, fetal, and EMG measurements. This embodiment allows for simultaneous monitoring of contraction, fetal heart rate (FHR) and maternal heart rate (MHR) and is thus suitable for hospital use during labor and delivery. Other electrode configurations (not shown) include providing an abdominal patch extending to the back of an expectant mother.

In the hospital and delivery embodiments of the invention, shown in FIG. 11, it is desirable to provide a wireless link 62 from the labor monitor patch 60 to an external monitor 65 to display monitored parameters sensed by the invented patch 60 during labor and delivery on the display unit 66 or on its printout 69. The wireless link 62, shown as RF signal, allows the mother to be ambulatory during labor while providing continuous uninterrupted data for the medical staff. Mobility is known to reduce stress for the mother during labor and may also shorten the duration of labor, which can be lengthy and very stressful for the mother and the baby. Ambulation during labor is problematic with current intrapartum monitoring instruments because the mother is typically confined to the bed with sensors attached to the mother on one end and a bedside monitor on the other end. The invented patch in the wireless embodiment provides a disposable electronic alternative, which is less expensive, more hygienic and less prone to loss of signal compared to conventional electromechanical sensors currently in use. Because these conventional sensors are reusable they require frequent cleaning and application of gel. Furthermore, movements of the mother and baby often necessitate repositioning of the sensors or adjustment of belt pressure for obtaining reliable signals. The electronic solution of the integrated patch eliminates positioning and movement problems while allowing the mother mobility, and is particularly suited for a lengthy labor.

EMG signals detected during contractions can be displayed by a standard fetal-maternal monitor 65 using standard toco input 67′, IUP input (not shown) and ultrasound input 68′ as shown in FIGS. 11 and 12. This is partially accomplished by providing an interface device 70 that produces an electrical signal 77 compatible with signal produced by a toco transducer or an IUP transducer. The interface device 70 comprises a wireless antenna 71, a wireless receiver/decoder 72, an amplifier 72, and a signal conditioner 76 for producing electrical signal 77 having a format and levels that emulate signals produced by standard pressure sensors. The contraction signal 77 is delivered through a standard toco plug 67, which feeds into toco input 67′, resulting in a standard contraction display 66 and printout 69 of the external monitor 65. This basically translates and correlates EMG activity, which is electrical in nature, to a pressure signal in mm HG that is standard and widely used and accepted. For example, a baseline of EMG activity at rest measured by the invented patch can be electronically correlated to a baseline display of approximately 10 mm Hg by producing the corresponding signal into the toco input 67′. On the other hand, an intense EMG burst activity can be electronically correlated to produce a display of 80 mm Hg on the display unit 66 of the external monitor 65. Similarly, fetal heart rate (FHR) information can be sent by the wireless patch 60 for receiving by the wireless decoder 72 and processing by the FHR amplifier 73 and FHR signal conditioner 74. The goal of the interface is to produce an FHR signal 78 delivered via the ultrasound plug 68 to the ultrasound input 68′ of the external monitoring instrument 65. The interface device 70 comprises a link indicator 79 to indicate proper wireless link when the invented patch 60 is detected in proximity. The interface box 70 is preferably powered by a power signal from the external monitor 65 via one of its ports, such as a toco input 67′, an ultrasound input 68′, an IUP input (not shown), or other ports available therein. This eliminates the need for a battery or separate power source for the interface device.

It should be obvious to those skilled in the art of medical electronics that other connection and input arrangements are possible for connecting the interface device or the invented patch to an external monitor in clinical setups. It should also be obvious that the contraction and FHR data should be of a suitable format, ranging from raw data to processed information ready for signal conditioning by the interface device 70. Furthermore, it should be obvious that the invented patch is not only suitable for monitoring labor in human females but also equally applicable to other mammals.

FIG. 7 is a schematic diagram that shows major components of a preferred embodiment comprising a reed-switch 39 (wireless sensor) incorporated in the patch device 10 for responding to a magnetic field from an external magnet (not shown) or programming device (not shown). In the programmable embodiment, the device can be configured with operational parameters according to the needs and condition of the expectant mother. Programming is preferably by wireless means incorporating a wireless sensor in the patch to receive coded wireless commands from an external transmitter (not shown).

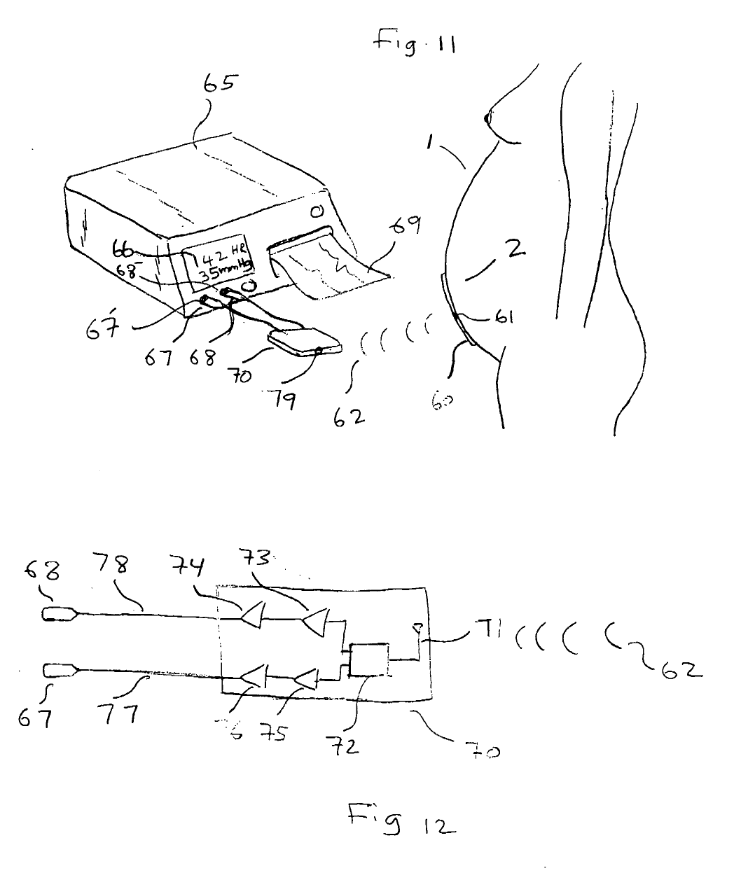

Other features can include the ability to store data in a memory 37 and transmit the stored data to a remote receiver, such as an external monitor 65 (FIG. 11) for display and clinical analysis by medical staff. FIG. 13 shows the acoustic trans-telephonic transmission of data via an audio transducer 44 incorporated within the patch device 10 to the mouthpiece of the telephone handset 85. In this embodiment, acoustic interrogation commands from the remote unit via the earpiece of the handset can also be downloaded into the patch device 10 via the receiver audio transducer 47.

The wireless reception of commands and transmission of data may be accomplished in numerous ways and methods known in the field of remote control and wireless transmission of medical data. This includes optical, radio frequency (RF), magnetic, ultrasonic, and acoustic transmission. The programming unit can also be incorporated in the receiver unit such as the interface device shown in FIG. 11. The combined controller/receiver unit can be in the form a desktop unit, a portable unit, or a handheld instrument. In one embodiment, an external monitor or a personal computer using a protocol, such as Medical Implants Communications Service (MICS), Wireless Medical Telemetry Services (WMTS), Blue Tooth or 802-11, and an appropriate software application as is known in the art can be used to receive and process signals from this device.

For detecting premature labor at home, the invented patch is preferably designed for long-term wear by the expectant mother. For this purpose, many design details should be incorporated for the device to function properly and reliably for extended periods of time exceeding several days or weeks. The adhesion to the abdomen skin may be designed for single-use or multiple applications. In single-use applications, the patch device is applied once for continuous wear until removed for its disposal. In this case, the patch is worn even during sleep and bathing. In multiple applications design, the adhesive allows for multiple removal and reapplication to the skin. In either design, the adhesive 55 incorporated in the device 10 must provide continuous reliable adhesion to prevent inadvertent peeling of the device from the abdomen skin. A biocompatible skin adhesive, such as hydrogel and like materials, has been shown to be effective in human skin applications. The ideal properties of the skin adhesive include being waterproof and air-permeable. Waterproof properties aid in the protection of the electrodes underneath from water-born contaminants. Air permeability properties allow for the healthy aeration of the tissue underneath the patch device.

To achieve longevity of operation for the patch device, various means for power conservation must be considered. This includes power management (PM) circuitry (24 FIG. 7) to shut off certain electronic components selectively when the device is not in use. The patch device 10 also incorporates stretchable areas 25 (FIG. 1) to allow for stretching and abdomen movements during motion, breathing, sleep, etc. The construction of the device must be durable and protective of the components within. Metal foil 38 (FIG. 4) covering the internal components and substrate 26 not only provides EMI protection, but also aids in water proofing and overall protection.

Proper patch adhesion to the skin is not only important for waterproofing purposes, but also to maintain proper electrode-skin contact throughout device wear and operation. This is important for obtaining adequate biopotential signal-to-noise-ratio.

Although the invention is described herein with reference to preferred embodiments, one skilled in the art will readily appreciate that other applications may be substituted for those set forth herein without departing from the spirit and scope of the present invention. Accordingly, the invention should only be limited by the Claims included below.

1. An apparatus for non-invasively monitoring and indicating a labor contraction of a pregnant female, comprising:
a wearable patch incorporating the following:
one or more sensors for receiving at least one vital sign signal present on the surface said pregnant female's body;
an amplifier for amplifying said vital sign signal from said sensors;
a power source for powering said integrated monitor patch;
a processor for analyzing and detecting said vital sign signal; and
means for extracting one or more vital signs representing labor contraction.

2. The apparatus of claim 1, wherein said vital sign signal is an EMG signal representing myometrial activity of said pregnant female.

3. The apparatus of claim 1, wherein said patch detects premature labor.

4. The apparatus of claim 1, wherein said patch is worn by a female having a high risk of pregnancy, wherein said female is prone to premature delivery.

5. The apparatus of claim 1, said patch detects one or more stages of labor.

6. The apparatus of claim 1, further comprising:
an indicator for indicating any of the presence of a contraction and a stage thereof.

7. The apparatus of claim 6, wherein said indicator produces a signal perceptible by the pregnant female wearing said integrated patch.

8. The apparatus of claim 6, wherein said indicator comprises a vibratory element for tactile perception by said pregnant female.

9. The apparatus of claim 1, wherein said one or more sensors comprise a biopotential electrode.

10. The apparatus of claim 9, wherein said electrode receives any of a fetal ECG signal and a maternal ECG signal.

11. The apparatus of claim 1, further comprising:
at least one electrode for positioning on the back of said pregnant female's lower abdomen.

12. The apparatus of claim 1, further comprising:
a memory for storing data representative of at least one vital sign.

13. The apparatus of claim 1, further comprising:
means for wireless transmission of at least one vital sign to a remote monitor device.

14. The apparatus of claim 13, wherein said wireless transmission is received by an interface device for receiving wireless signal from said patch and for delivering said signal to an external monitor.

15. An integrated patch for non-invasively detecting a premature labor contraction of a pregnant female wearing said patch on her body, said patch comprising:
at least two electrodes for receiving a myometrial EMG signal;
an amplifier for amplifying said EMG signal received from said electrodes;
a power source for powering said integrated patch;
a processor for analyzing and detecting an EMG pattern indicative of said premature labor contraction; and
an indicator activated by said processor upon detection of said EMG pattern that is indicative of premature labor contraction.

16. The integrated patch of claim 15, wherein said EMG pattern is characterized by epochs of intense short duration EMG bursts.

17. A system for non-invasively monitoring labor contraction of a pregnant female, comprising:
an integrated patch adhesively attached to, and worn on, the body of said pregnant female, said patch incorporating at least one biopotential electrode, a biopotential amplifier network for amplifying EHG sign present on said pregnant females's body during labor contraction, a power source for powering said monitor patch, and a wireless transmitter for sending labor contraction data representative of a myometrial contraction;
an interface device incorporating a wireless receiver for receiving labor contraction data from said integrated patch and a signal conditioner for producing a signal representative of myometrial activity; and
an external monitor device for displaying said labor contraction data received from said patch by said interface device.

18. The system of claim 17, wherein said external monitor device comprises a standard electronic maternal-fetal monitor device having a standard toco input port.

19. The system of claim 17, wherein said signal conditioner produces a signal representing a simulated pressure signal from a toco transducer.

20. A method of non-invasive monitoring of myometrial activity of a pregnant female, comprising the steps of;
adhesively attaching an integrated patch on the abdomen area of said pregnant female, said patch comprising one or more myometrial activity sensors, one or more amplifiers for amplifying a signal from said one or more sensors, a processor, and a power source;
amplifying said myometrial sensor signal obtained from said one or more sensors by said one or more amplifiers;
analyzing said amplified myometrial signal and searching for myometrial signal patterns indicative of labor contractions with said processor.

21. The method of claim 20, further comprising the step of:
indicating the occurrence of a contraction.

22. The method of claim 21 wherein said step of indicating the occurrence of a contraction is executed by an indicator incorporated in said integrated patch.

23. A method for non-invasive detection of labor for a pregnant female, comprising the steps of:
adhesively attaching an integrated patch on the abdomen area of said pregnant female, said patch comprising a thin flexible substrate, at least two EMG electrodes contacting the skin of said pregnant female at the abdomen area for receiving EMG signals, an EMG amplifier, a power source, a processor, and an indicator;
amplifying said EMG signal obtained from said EMG electrodes by said amplifier;
analyzing said amplified EMG signal with said processor;
detecting EMG patterns indicative of a labor contraction; and
activating said indicator by said processor to alert for the presence of a contraction.

24. The method of claim 23, wherein said indicator comprises a vibratory element perceptible by said pregnant female when said indicator is activated.

25. A method for non-invasive monitoring of labor for a pregnant female, comprising the steps of:
adhesively attaching an integrated patch on the abdomen area of said pregnant female, said patch comprising a thin flexible substrate, at least two EMG electrodes contacting the skin of said pregnant female at the abdomen area for receiving EMG signal, an EMG amplifier, and a wireless transmitter;
amplifying said EMG signal obtained from said EMG electrodes with said amplifier;
sending wireless signal representing said EMG signal indicative of labor contractions by said wireless transmitter to an external monitor; and
displaying labor contraction data with said external monitor.

26. The method of claim 25, further comprising the step of:
sending said wireless signal is sent to said external monitor via an interface device comprising a wireless receiver and a signal conditioner for producing electrical signal compatible with a signal produced by any of a toco transducer and an IUP transducer.
